(19) United States
(12) Patent Application Publication
(10) Pub. No.: US 2005/0075572 A1
(43) Pub. Date: Apr. 7, 2005

(54) Focusing micromachined ultrasonic transducer arrays and related methods of manufacture

(76) Inventors: David M. Mills, Niskayuna, NY (US); Douglas G. Wildes, Ballston Lake, NY (US); Charles E. Baumgartner, Niskayuna, NY (US); Lowell Scott Smith, Niskayuna, NY (US); Robert S. Lewandowski, Amsterdam, NY (US)

Correspondence Address: Dennis M. Flaherty, Esq.;Ostrager Chong & Flaherty LLP, 825 Third Avenue, 30th Floor, New York, NY, 10022-7519, US

(21) Appl. No.: 10/676,202

(22) Filed: Oct. 1, 2003

(51) Int. Cl. A61B 8/14

(52) U.S. Cl. 600/459

(57) ABSTRACT

A micromachined ultrasonic transducer array that focuses in the elevation direction. A curved lens is used to narrow the beam width in the elevation direction so that contrast resolution is improved and clinically relevant. Alternatively, a curved probe is formed by bending a micromachined substrate to have a predetermined curvature. The invention is further directed to methods of manufacturing such transducers.

### BACKGROUND OF THE INVENTION

This invention generally relates to arrays of micromachined ultrasonic transducers (MUTs). One specific application for MUTs is in medical diagnostic ultrasound imaging systems.

Conventional ultrasound imaging systems comprise an array of ultrasonic transducers that are used to transmit an ultrasound beam and then receive the reflected beam from the object being studied. Such scanning comprises a series of measurements in which the focused ultrasonic wave is transmitted, the system switches to receive mode after a short time interval, and the reflected ultrasonic wave is received, beamformed and processed for display. Typically, transmission and reception are focused in the same direction during each measurement to acquire data from a series of points along an acoustic beam or scan line. The receiver is continuously refocused along the scan line as the reflected ultrasonic waves are received.

For ultrasound imaging, the array typically has a multiplicity of transducers arranged in one or more rows and driven with separate voltages in transmit. By selecting the time delay (or phase) and amplitude of the applied voltages, the individual transducers in a given row can be controlled to produce ultrasonic waves that combine to form a net ultrasonic wave that travels along a preferred vector direction and is focused in a selected zone along the beam.

The same principles apply when the transducer probe is employed to receive the reflected sound in a receive mode. The voltages produced at the receiving transducers are summed so that the net signal is indicative of the ultrasound reflected from a single focal zone in the object. As with the transmission mode, this focused reception of the ultrasonic energy is achieved by imparting separate time delay (and/or phase shifts) and gains to the signal from each receiving transducer. The time delays are adjusted with increasing depth of the returned signal to provide dynamic focusing on receive.

The quality or resolution of the image formed is partly a function of the number of transducers that respectively constitute the transmit and receive apertures of the transducer array. Accordingly, to achieve high image quality, a large number of transducers, referred to herein as elements, is desirable for both two- and three-dimensional imaging applications. The ultrasound elements are typically located in a hand-held transducer probe that is connected by a flexible cable to an electronics unit that processes the transducer signals and generates ultrasound images. The transducer probe may contain both ultrasound transmit circuitry and ultrasound receive circuitry.

Recently semiconductor processes have been used to manufacture ultrasonic transducers of a type known as micromachined ultrasonic transducers (MUTs), which may be of the capacitive (cMUT) or piezoelectric (pMUT) variety. cMUTs are tiny diaphragm-like devices with electrodes that convert the sound vibration of a received ultrasound signal into a modulated capacitance. For transmission the capacitive charge is modulated to vibrate the diaphragm of the device and thereby transmit a sound wave.

One advantage of MUTs is that they can be made using semiconductor fabrication processes, such as microfabrication processes grouped under the heading “micromachining”. As explained in U.S. Pat. No. 6,359,367:
Micromachining is the formation of microscopic structures using a combination or subset of (A) Patterning tools (generally lithography such as projection-aligners or wafer-steppers), and (B) Deposition tools such as PVD (physical vapor deposition), CVD (chemical vapor deposition), LPCVD (low-pressure chemical vapor deposition), PECVD (plasma chemical vapor deposition), and (C) Etching tools such as wet-chemical etching, plasma-etching, ion-milling, sputter-etching or laser-etching. Micromachining is typically performed on substrates or wafers made of silicon, glass, sapphire or ceramic. Such substrates or wafers are generally very flat and smooth and have lateral dimensions in inches. They are usually processed as groups in cassettes as they travel from process tool to process tool. Each substrate can advantageously (but not necessarily) incorporate numerous copies of the product. There are two generic types of micromachining . . . 1) Bulk micromachining wherein the wafer or substrate has large portions of its thickness sculptured, and 2) Surface micromachining wherein the sculpturing is generally limited to the surface, and particularly to thin deposited films on the surface. The micromachining definition used herein includes the use of conventional or known micromachinable materials including silicon, sapphire, glass materials of all types, polymers (such as polyimide), polysilicon, silicon nitride, silicon oxynitride, thin film metals such as aluminum alloys, copper alloys and tungsten, spin-on-glasses (SOGs), implantable or diffused dopants and grown films such as silicon oxides and nitrides.
   The same definition of micromachining is adopted herein. The systems resulting from such micromachining processes are typically referred to as “micromachined electro-mechanical systems (MEMS).

A typical cMUT cell comprises a thin silicon or silicon nitride membrane with an overlying metal electrode, suspended over a cavity formed on a silicon substrate. A bottom electrode is formed in or on the silicon substrate or by doping the substrate so that it is conductive. All cMUT cells in an element are electrically connected using hard-wired top and bottom electrodes. The membrane vibrates to both emit and receive ultrasonic waves. The driving force for the deflection of the membrane during transmit is the electrostatic attraction between the top and bottom electrodes when a voltage is impressed across them. If an alternating voltage drives the membrane, significant ultrasound generation results. Conversely, if the membrane is biased appropriately and subjected to incoming ultrasonic waves, significant detection currents are generated. Typical thicknesses of the membrane lie in the range of 1 to 3 microns and the cavity gap is on the order of 0.1 to 0.3 micron. The lateral dimensions of the cMUT cell range from 10 to 100 microns for cMUT array operating frequencies of 2 to 15 MHz.

The cMUT (or pMUT) cells can be arranged and hard-wired to form a row of elements. Ultrasound energy emitted from such a linear cMUT array is too broad in the elevation direction to be useful for medical imaging. There is a need for MUT arrays that are more narrowly focused in the elevation direction. BRIEF DESCRIPTION OF THE INVENTION

The present invention is directed to micromachined ultrasonic transducer arrays that focus in the elevation direction. A curved lens is used to narrow the beam width in the elevation direction so that contrast resolution is improved and clinically relevant. Alternatively, a curved probe is formed by bending a micromachined substrate to have a predetermined curvature. The invention is further directed to methods of manufacturing such transducers.

One aspect of the invention is an elevationally focused ultrasonic probe comprising an array of MUT cells. In accordance with one embodiment, the probe further comprises a curved lens adhered to the array of MUT cells, and a planar substrate, the MUT cells being built on the substrate. In accordance with another embodiment, the probe further comprises a curved substrate, the MUT cells being built on the substrate, and a layer of protective material covering the array of MUT cells.

Another aspect of the invention is an ultrasonic probe comprising: a curved substrate having a profile that is substantially constant in an azimuthal direction; an array of MUT cells built on the curved substrate and facing toward a line of focus, the MUT cells being disposed on a concave side of the curved substrate; and a layer or protective material of generally constant thickness applied on the face of the array of MUT cells.

A further aspect of the invention is a lensing process comprising the following steps: (a) micromachining an array of ultrasonic transducer cells on a substrate; (b) applying a layer of adhesive material on a preformed curved lens or on a surface of the micromachined substrate; (c) placing the lens in abutment with the micromachined substrate with the layer of adhesive material therebetween; and (d) curing the adhesive material.

Yet another aspect of the invention is a lensing process comprising the following steps: (a) micromachining an array of ultrasonic transducer cells on a substrate; (b) casting lensing material on a surface of the micromachined substrate; and (c) curing the lensing material.

Another aspect of the invention is a method of making a curved ultrasonic probe, comprising the following steps: (a) micromachining an array of ultrasonic transducer cells on a substrate; and (b) bending the substrate to have a predetermined curvature suitable for focusing ultrasonic beams emitted by the array in an elevational direction.

A further aspect of the invention is an integrated device comprising: a curved lens; a first multiplicity of MUT cells hard-wired together and disposed underneath the lens; a second multiplicity of MUT cells hard-wired together and disposed underneath the lens; CMOS electronics disposed underneath the first and second multiplicities of MUT cells; and a silicon substrate disposed underneath the CMOS electronics.

Other aspects of the invention are disclosed and claimed below.

### BRIEF DESCRIPTION OF THE DRAWINGS

FIG. 1 is a drawing showing a cross-sectional view of a typical cMUT cell.

FIG. 2 is a drawing showing hexagonal MUT cells of an element in accordance with one embodiment of the invention.

FIG. 3 is a drawing showing an isometric view of a micromachined structure comprising a one-dimensional or 1.5 dimensional array of transducer elements made from cMUT cells.

FIG. 4 is a drawing showing an isometric view of a lensed micromachined structure comprising a cylindrical lens in accordance with a first embodiment of the invention.

FIG. 5 is a drawing showing an isometric view of a lensed micromachined structure comprising a multifocal lens in accordance with a second embodiment of the invention.

FIG. 6 is a drawing showing an isometric view of a lensed micromachined structure comprising an elliptical lens in accordance with a third embodiment of the invention.

FIG. 7 is a drawing showing the different layers of a lensed micromachined structure in accordance with one or more of the disclosed embodiments.

FIG. 8 is a drawing showing the structure of a curved cMUT array in accordance with another embodiment of the invention.

Reference will now be made to the drawings in which similar elements in different drawings bear the same reference numerals.

### DETAILED DESCRIPTION OF THE INVENTION

The innovation disclosed here is a unique method of implementing an array with micromachined ultrasound transducers (MUTs). For the purpose of illustration, various embodiments of the invention will be described that utilize capacitive micromachined ultrasonic transducers (cMUTs). However, it should be understood that the aspects of the invention disclosed herein are not limited to use of cMUTs, but rather may also employ pMUTs.

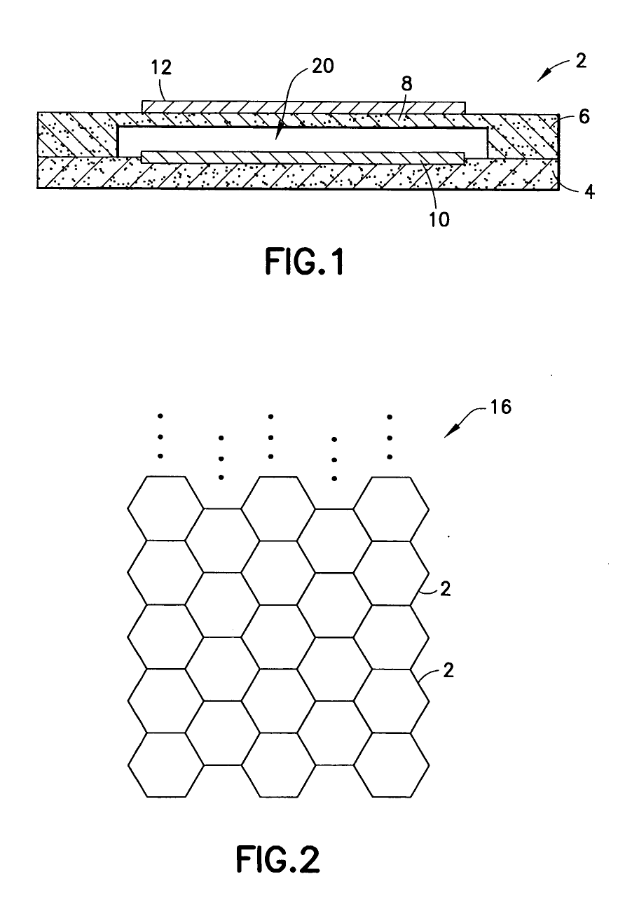

cMUTs are silicon-based devices that comprise small (e.g., 50 μm) capacitive “drumheads” or cells that can transmit and receive ultrasound energy. Referring to FIG. 1, a typical MUT transducer cell 2 is shown in cross section. An array of such MUT transducer cells is typically fabricated on a substrate 4, such as a silicon wafer. For each MUT transducer cell, a thin membrane or diaphragm 8, which may be made of silicon, silicon nitride, or other suitable material, is suspended above the substrate 4. The membrane 8 is supported on its periphery by an insulating support 6, which may be made of silicon, silicon oxide, or silicon nitride. The cavity 15 between the membrane 8 and the substrate 4 may be air- or gas-filled or wholly or partially evacuated. A film or layer of conductive material, such as aluminum alloy or other suitable conductive material, forms an electrode 12 on the membrane 8, and another film or layer made of conductive material forms an electrode 10 on the substrate 4. Alternatively, the electrode 10 can be embedded in the substrate 4 or substrate 4 may itself be conductive, such as doped n- or p-type silicon. Also the electrode 12 can be on top of membrane 8 rather than embedded within it as shown in FIG. 1.

The two electrodes 10 and 12, separated by the cavity 15, form a capacitance. When an impinging acoustic signal causes the membrane 8 to vibrate, the variation in the capacitance can be detected using associated electronics (not shown in FIG. 1), thereby transducing the acoustic signal into an electrical signal. Conversely, an AC signal applied to one of the electrodes will modulate the charge on the electrode, which in turn causes a modulation in the capacitive force between the electrodes, the latter causing the diaphragm to move and thereby transmit an acoustic signal.

In one mode of operation, the cMUT cell typically has a dc bias voltage V_{bias} that is significantly higher than the time-varying voltage v(t) applied across the electrodes. The bias attracts the top electrode toward the bottom through coulombic force. In this heavily biased case, the cMUT drumheads experience a membrane displacement u given as follows:

$$u ( t ) \approx \frac{ɛ}{{d}^{2}} * {V}_{bias} * v ( t ) \qquad ( 1 )$$

where d is the distance between the electrodes or plates of the capacitor, and a is the effective dielectric constant of the cell. The sensitivity of the cMUT cell has been found to be the greatest when the bias voltage is high and electrodes are closer together.

In the above-described mode, the membrane is biased up to, but not into collapse. Deflection of the membrane is limited to about ⅔ of the original gap. (i.e., the membrane can deflect only ⅓ of the gap before something else happens). Some recent work focuses on a so-called “collapse mode” wherein the center of the membrane has jumped across the gap and is actually touching the substrate. The part of the membrane that is not touching the substrate then experiences a much smaller gap, and higher sensitivity. Lensing, as disclosed herein, would apply equally to either mode of operation.

Due to the micron-size dimensions of a typical MUT, numerous MUT cells are typically fabricated in close proximity to form a single transducer element. The individual cells can have round, rectangular, hexagonal, or other peripheral shapes. Hexagonal shapes provide dense packing of the MUT cells of a transducer element. The MUT cells can have different dimensions so that the transducer element will have composite characteristics of the different cell sizes, giving the transducer a broadband characteristic.

MUT cells can be hard-wired together to form elements. A portion of one element 16 having five columns of cells 2, the columns extending as far as necessary to fill the given element size, is shown in FIG. 2.

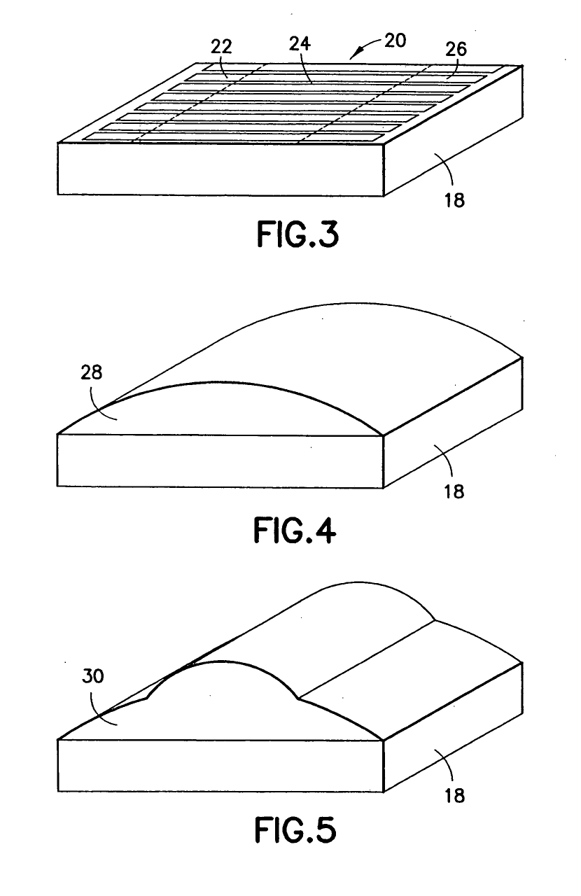

A design for a linear array of elements made up of cMUT cells is generally depicted in FIG. 3. A multiplicity of cMUT cells are built on a CMOS wafer 18 using micromachining techniques. The cMUT cells are arranged to form a single row of ultrasonic transducer elements 20 arrayed in an azimuthal direction, each element 20 covering a generally rectangular area with the elements arrayed in a side-by-side relationship. The cMUT cells of each element 20 are connected in parallel. Each element 20 may comprise 100 to 1,000 cMUT cells (really this is any number that is necessary to fill the given element size). For example, an element may comprise a multiplicity of hexagonal cells arranged in six rows, each row having on the order of 100 cells and being generally aligned with an elevational direction. The cMUT cells of an element all resonate together to generate an ultrasound wavefront.

To provide a 1.5-dimensional transducer array, each rectangular region may be divided into three generally rectangular sub-regions 22, 24, and 26, as indicated by dashed lines in FIG. 3. The lengths of sub-regions 22 and 26 are equal and typically (but not necessarily) less than the length of the central sub-region 24. In accordance with this alternative embodiment, the cMUT cells in sub-region 22 are hard-wired together; the cMUT cells in sub-region 24 are hard-wired together; and the cMUT cells in sub-region 26 are hard-wired together, thus forming three elements in each column. In this case, the cMUT cells of sub-regions 22 and 26 are preferably activated concurrently during transmission, but in some configurations they can be independent.

Naturally, the foregoing concept can be extrapolated to build probes having more than three rows of transducer elements.

Instead of hard-wiring the cMUT cells to form elements, they may be hard-wired to form sub-elements, which sub-elements are then interconnected by switches (integrated into the CMOS wafer) to form elements.

In accordance with various embodiments of the invention depicted in FIGS. 4-6, a focused probe, comprising cMUT cells microfabricated on a CMOS wafer 18, is created by attaching a curved lens to the front faces, i.e., the membranes, of the cMUT cells. The purpose of focusing the ultrasonic probe is to limit the thickness of the plane that is interrogated by the ultrasound energy. However, since the membranes are delicate, care must be taken during lensing to not damage the cMUT cells.

In the embodiment depicted in FIG. 4, a cylindrical lens 28 is attached to the front face of the cMUT cell array. The axis of the cylinder is parallel to the azimuthal direction of the array. The cylindrical lens is typically used when the substrate has only a single row of transducer elements.

FIG. 5 shows a multifocal lens 30 attached to the front faces of the MUT cell array. This multifocal lens 30 is used in conjunction with a transducer array comprising three rows of elements, as indicated by the dashed lines in FIG. 3. The central focal zone of the multifocal lens 30 overlies the central sub-region 24, seen in FIG. 3. A multifocal lens allows for multiple focal regions.

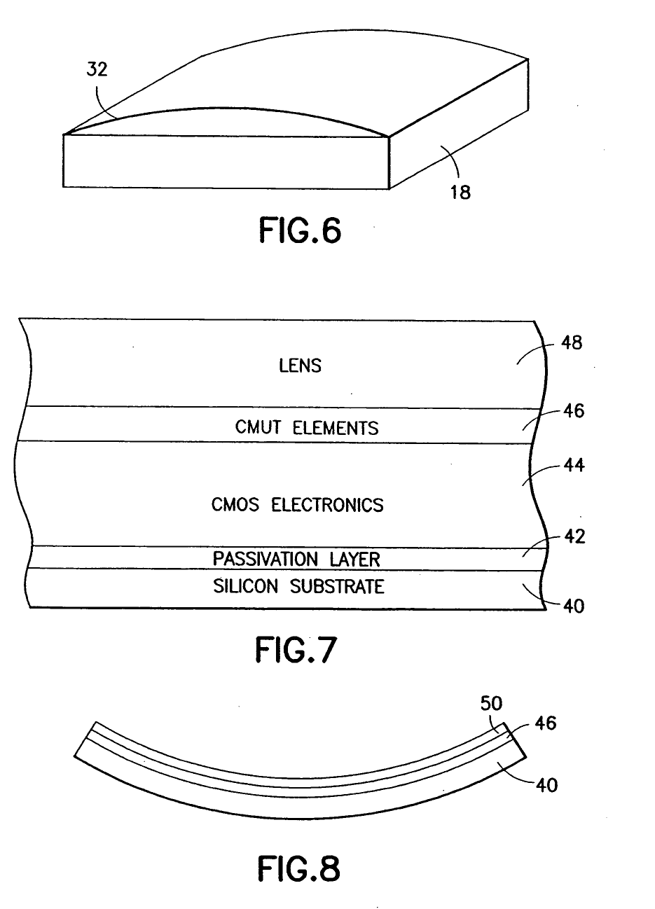

In accordance with a further embodiment of the invention depicted in FIG. 6, an elliptical lens 32 is attached to the front face of the cMUT cell array. The axis of the elliptical lens is parallel to the azimuthal direction of the array. The elliptical lens is also typically used when the substrate has only a single row of transducer elements. The elliptical lens (and other acylindrical lenses) eliminates cylindrical lens aberrations.

Care must be taken when selecting the material to use for the lens as the characteristics of the lens are important to obtaining a robust ultrasound probe. Acoustically, it must have a similar impedance, the product of density and speed of sound in the material, to that of water so as to avoid sound reflections at the lens/water interface. It must also have a different speed of sound than water, preferably lower than water, so as to focus the acoustic wavefront in the elevation plane. The acoustic attenuation of the lens must also be low to maximize the transmission of sound through the lens and minimize heating. Chemical properties, such as water permeability, must also be carefully selected to keep chemicals away from the cMUT cells. The lens must also be durable so that it can be used many times without tearing or cracking due to material fatigue. Representative lens materials include silicone rubbers like GE RTV 60, RTV 560 and RTV 630.

The polymeric material of the lens must be sufficiently flexible to allow the attached cMUT cells to vibrate. An experiment was performed to determine whether bonding lens material to the face of a cMUT cell array would be possible without destroying the membranes and severely reducing the sensitivity of the device. This preliminary study demonstrated that the membranes were still intact and acoustically active after bonding.

A high-bandwidth cMUT array can be integrated with conventional CMOS switches and preamplifier/buffer circuits on a silicon wafer to provide reconfigurable beam-forming elements. In such an integrated structure, the size of the transducer subelements establishes the dimensions of the cells for the microelectronics in the silicon immediately below the array. This integrated structure is generally depicted in FIG. 7. A passivation layer 42 (made, e.g., of oxide) is placed on top of a silicon substrate 40. CMOS electronics 44 are fabricated on top of the passivation layer. Then the cMUT subelements 46 are fabricated on top of the CMOS electronics. A lens 48 is attached to the front faces of the cMUT subelements.

The cMUT bottom electrodes (not shown in FIG. 7) are connected to the CMOS electronics using an additional metal level (not shown) in the cMUT fabrication process. This metal level comprises metal plugs through the CMOS dielectric passivation layers (not shown). The CMOS electronics are integrated underneath each cMUT subelement. The integrated electronics comprise high-voltage switching transistors, gate-drive transistors for the switches, and control logic and buffers. A high-voltage switch is configured to connect each subelement to its bias voltage and another high-voltage switch connects each subelement to an adjacent subelement to form an array element.

While applying a lens to focus a conventional piezoelectric ultrasound transducer array is known, using a lens to focus a MEMS device is not. The surface of a MEMS device is delicate and may consist of several different materials such as silicon, silicon oxide, silicon nitride, and/or metals such as gold or aluminum. The procedure used to attach a preformed lens to the MEMS surface, therefore, must be capable of developing adhesion to several different materials simultaneously while not causing any chemical or mechanical damage to the device surface.

One example of a method useful for bonding a formed silicone lens to a MEMS device comprises the following steps: (1) cleaning of both surfaces; (2) applying an adhesion promoter to one or both surfaces; (3) adhering the lens to the MEMS device using an appropriate adhesive such as a low-viscosity RTV silicone; and (4) using a low applied pressure to contact the adhesive to both surfaces while the adhesive cures.

Several methods are available for cleaning the lens and MEMS surfaces. The preferred cleaning technique is exposing the surfaces to an oxygen-containing plasma as this technique does not possess the potential for damage to the MEMS device. Others include scrubbing with an ultrasonic cleaner and a suitable non-ionic surfactant or rinsing with a solvent such as isopropyl alcohol or acetone

The adhesion promoter should be of a generic nature to allow adhesion to the adhesive and to a variety of different surfaces. In the case of an RTV adhesive, useful adhesion promoters are selected from the general class of silicates, such as tetraethylsilicate, organometallics, such as organotitanates, and reactive organosilanes, such as organomethoxysilanes.

It is preferable that the adhesive possess a low viscosity, so as to be applied in a thin layer to the device surface, and that the adhesive be an elastomer so that it does not damage the MEMS surface. A low pressure, i.e., less than 50 psi and preferably less that 10 psi, is used to cause contact between the MEMS surface and the lens surface while leaving a thin layer of adhesive intact.

One specific example for attaching a focused silicone lens to a MEMS device uses an oxygen-containing plasma to clean both surfaces, a solution of 1% by weight of tetraethylsilicate in alcohol applied to both the surface of the lens and the surface of the MEMS device as an adhesion promoter, an RTV silicone as an adhesive, and curing of the adhesive with an external pressure of 3 psi applied to the exterior of the lens to force contact of the inner lens surface and the MEMS top surface.

In accordance with a further embodiment, a layer of Parylene is vapor deposited directly onto the MEMS surface before the lens is applied to provide a barrier to prevent chemicals that diffuse through the silicone lens from reaching the surface of the MEMS device. Alternatively, metal could be sputtered onto the MEMS surface to prevent chemical diffusion.

A DOE evaluating cMUT lens bonding using a silicone adhesion promoter, comprised primarily of tetraethylorthosilicate in an aliphatic hydrocarbon solvent and an RTV silicone adhesive was performed. The lens was bonded to the wafer using the lensing procedure outlined below.

A. Wafer Preparation:

(1) Plasma clean the wafer surface in 2% O₂/Ar.

(2) Soak in adhesion promoter solution prepared by mixing SS-4155 in isopropanol alcohol. The adhesion promoter concentration and soak time were variables in the experimental design.

(3) Blow off excess adhesion promoter solution with N₂ and dry for 20 minutes in a 50° C. oven.

B. Silicone Lens Material Preparation:

(1) Immerse in 65° C. aqueous solution containing 2% non-ionic surfactant for 2 minutes with ultrasonic agitation. Rinse in flowing de-ionized water for 2 minutes.

(2) Blow off water with N₂ and dry for 20 minutes in a 50° C. oven.

(3) The application of adhesion promoter (as above) to the silicone surface was one of the experimental design variables.

C. Bonding Process:

(1) Silicone adhesive KE-1604 was combined with the curative catalyst at a ratio of 10 to 1. The mixed adhesive was degassed for 10 minutes under vacuum.

(2) A thin layer of adhesive was applied to the surface of the silicone peel strip and placed onto the wafer. The application of a 10 psi pressure and the use of a 50° C. temperature during the initial 16 hour cure process were variables in the experimental design.

The DOE results showed that application of SS-4155 adhesion promoter to both interfaces and initial curing of the KE-1604 adhesive at 50° C. to be significant variables for increasing peel adhesion. Other factors were not significant and no significant interactions were found. Adhesion increased for all samples with 10-day storage at ambient temperature.

Applying the SS-4155 adhesion promoter to only the wafer surface and curing the sample at room temperature yielded an average peel adhesion after a 16-hour cure of 0.9 pli (pounds/linear inch width), increasing to 2.5 pli after 10 days. In contrast, treating both interfaces with adhesion promoter and curing the samples at 50° C. yielded a 4.1 pli adhesion after 16 hours, increasing to 5.8 pli after 10 days at ambient temperature.

In cases where the lens is cast directly on the face of the cMUT array, the surfaces of the cMUTs are cleaned and treated with adhesion promoter, but adhesive is not needed.

In yet another embodiment of this invention, the MEMS device itself is curved to focus the ultrasound energy at a desired focal depth. This may be accomplished by building the MEMS device on a flexible substrate, or by either building the MEMS device on a thin silicon substrate or by thinning the backside of the silicone substrate following MEMS fabrication.

The structure of an exemplary curved cMUT array is presented in FIG. 8. A multiplicity of cMUT subelements 46 are supported by a silicon substrate 40. The front faces of the cMUT cells are covered by a layer 50 of protective material without focusing characteristics (i.e., a protective layer of substantially constant thickness or the same speed of sound as water or the tissue being imaged). The protective layer 50 protects the cMUTs from external shock due to dropping, sharp instruments, or any other mechanical stress that may puncture membranes or in some other way damage the device. In addition, external environmental conditions, such as water, sterilizing liquids, or other fluids that may be applied to the surface of the probe, could damage the device. The protective layer 50 also protects the cMUT subelements 46 from these conditions. Attachment of the protective layer can be by casting it directly on the cMUT array or by bonding a preformed protective layer to the face of the cMUT array.

The method for fabricating the curved array shown in FIG. 8 is as follows. First, a multiplicity of cMUT subelements is microfabricated on a silicon substrate 40. Either the silicon substrate is thin enough to bend or the substrate is thinned after the CMUTs have been fabricated. The micromachined substrate is then bent around a form factor, with the cMUTs disposed on the concave surface abutting the form factor. In this curved state, epoxy is applied on the bottom of the silicon substrate and then cured. The cured epoxy holds the substrate in the curved state. A layer of protective material is then attached to the concave face of the cMUT array, which may have a constant thickness as in FIG. 8, or may have a top surface that is flat and a bottom surface that follows the curvature of the array (as long as the speed of sound in the protective material is generally equal to the speed of sound in water or the tissue being imaged, e.g., urethane).

As shown above, ultrasound energy from a MEMS device can be focused to a thinner plane in the elevation direction. Better contrast resolution for medical imaging results when the interrogated plane is thinner, which results in better diagnosis of injury or disease. Also, greater energy is delivered to the region of interest and this results in greater depth penetration for a given frequency. Also, the lens may act as a protective layer applied to the face of a MEMS transducer array to prevent mechanical damage to the device and to prevent exposure of high voltage to the outside (i.e., a patient in medical ultrasound applications).

In addition to use in medical imaging, the types of transducer arrays disclosed hereinabove could also be used in the area of non-destructive testing for materials such as metal forgings, turbine blades, nuclear reactors, oil pipelines, etc., where the array is used to inspect a material for cracks or other defects that do not show up optically or cannot be seen for other reasons.

While the invention has been described with reference to preferred embodiments, it will be understood by those skilled in the art that various changes may be made and equivalents may be substituted for elements thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

1. An elevationally focused ultrasonic probe comprising an array of MUT cells.

2. The probe as recited in claim 1, further comprising a curved lens adhered to said array of MUT cells, and a planar substrate, said MUT cells being built on said substrate.

3. The probe as recited in claim 2, further comprising a layer of adhesive material between said lens and said array.

4. The probe as recited in claim 3, further comprising a barrier layer disposed between said layer of adhesive material and said array of MUT cells, said barrier layer being made of a material that prevents chemical diffusion from said lens to said MUT cells.

5. The probe as recited in claim 1, further comprising a curved substrate, said MUT cells being built on said substrate, and a layer of protective material covering said array of MUT cells.

6. The probe as recited in claim 1, wherein said array comprises a first multiplicity of MUT cells hard-wired together and a second multiplicity of MUT cells hard-wired together.

7. The probe as recited in claim 6, wherein said MUT cells of said first multiplicity are arranged side by side and cover a generally rectangular area, the length of said rectangle being aligned with an elevation direction, said lens being curved in said elevation direction.

8. The probe as recited in claim 1, wherein said lens is cylindrical, multifocal or elliptical.

9. The probe as recited in claim 2, further comprising adhesion promoting material applied on a front face of said array or on a rear face of said lens or both.

10. The probe as recited in claim 5, further comprising adhesion promoting material applied on a front face of said array or on a surface of said protective layer that faces said array.

11. The probe as recited in claim 2, wherein said lens is made of a polymeric material.

12. The probe as recited in claim 11, wherein said lens is made of silicone rubber and said adhesive material is made of room-temperature vulcanizing silicone rubber.

13. The probe as recited in claim 9, wherein said adhesion-promoting material is a silicate.

14. The probe as recited in claim 9, wherein said adhesion-promoting material is an organometallic.

15. The probe as recited in claim 9, wherein said adhesion-promoting material is a reactive organosilane.

16. The probe as recited in claim 1, wherein each of said MUT cells is a capacitive MUT cell.

17. The probe as recited in claim 1, wherein each of said MUT cells is a piezoelectric MUT cell.

18. The probe as recited in claim 1, further comprising:
a layer of CMOS electronics below said array of MUT cells; and
a silicon substrate below said layer of CMOS electronics.

19. An ultrasonic probe comprising:
a curved substrate having a profile that is substantially constant in an azimuthal direction;
an array of MUT cells built on said curved substrate and facing toward a line of focus, said MUT cells being disposed on a concave side of said curved substrate; and
a layer or protective material applied on the face of said array of MUT cells, said layer having a substantially constant thickness or has a flat top surface and a bottom surface that follows the curvature of the substrate, if the speed of sound in the protective material is generally equal to the speed of sound in water or tissue.

20. The probe as recited in claim 19, wherein each of said MUT cells is a capacitive MUT cell.

21. The probe as recited in claim 19, wherein each of said MUT cells is a piezoelectric MUT cell.

22. A lensing process comprising the following steps:
(a) micromachining an array of ultrasonic transducer cells on a substrate;
(b) applying a layer of adhesive material on a preformed curved lens or on a surface of said micromachined substrate;
(c) placing said lens in abutment with said micromachined substrate with said layer of adhesive material therebetween; and
(d) curing said adhesive material.

23. The process as recited in claim 22, further comprising the step of applying a low pressure less than 50 psi during curing.

24. The process as recited in claim 22, wherein curing occurs at a temperature higher than room temperature.

25. The process as recited in claim 22, further comprising the step, performed before step (c), of cleaning the surfaces of said lens and said micromachined substrate that will be in contact with the adhesive material.

26. The process as recited in claim 25, wherein said cleaning step comprises exposing said surfaces to an oxygen-containing plasma.

27. The process as recited in claim 22, further comprising the steps, performed before step (c), of applying a layer of adhesion-promoting material to one or both of the surfaces of said lens and said micromachined substrate that will be in contact with the adhesive material.

28. The process as recited in claim 27, wherein said adhesion-promoting material is a silicate.

29. The process as recited in claim 27, wherein said adhesion-promoting material is an organometallic.

30. The process as recited in claim 27, wherein said adhesion-promoting material is a reactive organosilane.

31. The process as recited in claim 22, further comprising the step, performed before step (b), of depositing a layer of barrier material on the surface of said micromachined substrate that will be in contact with the adhesive material, said barrier material having the property of preventing chemical diffusion into said micromachined substrate.

32. A lensing process comprising the following steps:
(a) micromachining an array of ultrasonic transducer cells on a substrate;
(b) casting lensing material on a surface of said micromachined substrate; and
(c) curing said lensing material.

33. The process as recited in claim 32, further comprising the step, performed before step (b), of cleaning the surface of said micromachined array.

34. The process as recited in claim 33, further comprising the steps of applying a layer of adhesion-promoting material to said cleaned surface.

35. The process as recited in claim 34, further comprising the step of depositing a layer of barrier material on the surface of said micromachined array, said barrier material having the property of preventing chemical diffusion into said micromachined substrate.

36. A method of making a curved ultrasonic probe, comprising the following steps:
(a) micromachining an array of ultrasonic transducer cells on a substrate; and
(b) bending said substrate to have a predetermined curvature suitable for focusing ultrasonic beams emitted by said array in an elevational direction.

37. The method as recited in claim 36, further comprising the step of thinning said substrate after step (a) and before step (b).

38. The method as recited in claim 36, further comprising the step of applying a layer of protective material on a face of said array.

39. An integrated device comprising:
a curved lens;
a first multiplicity of MUT cells hard-wired together and disposed underneath said lens;
a second multiplicity of MUT cells hard-wired together and disposed underneath said lens;
CMOS electronics disposed underneath said first and second multiplicities of MUT cells; and
a silicon substrate disposed underneath said CMOS electronics.

40. The device as recited in claim 39, wherein each of said MUT cells is a capacitive MUT cell.

41. The device as recited in claim 39, wherein each of said MUT cells is a piezoelectric MUT cell.
